(19) United States
(12) Patent Application Publication
(10) Pub. No.: US 2005/0096283 A1
(43) Pub. Date: May 5, 2005

(54) Methods of inhibiting fertility

(75) Inventors: Maria Patricia Morales-Levy, Durham, NC (US); Irwin K.M. Liu, Winters, CA (US); Gary H. Rhodes, Winters, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of University of California, Oakland, CA (US)

(21) Appl. No.: 10/672,407

(22) Filed: Sep. 26, 2003

Related U.S. Application Data
(63) Continuation-in-part of application No. 10/313,463, filed on Dec. 6, 2002.
(60) Provisional application No. 60/340,141, filed on Dec. 14, 2001.

(51) Int. Cl. A61K 48/00

(52) U.S. Cl. 514/44

(57) ABSTRACT

The present invention features compositions and methods for inhibiting fertility or inducing contraception in a mammal. The compositions comprise a nucleic acid molecule, typically an immunostimulatory nucleic acid.

### CROSS REFERENCE TO RELATED APPLICATIONS

This application is a continuation in part of U.S. Ser. No. 10/313,463, file Dec. 6, 2002, which claims benefit of U.S. Ser. No. 60/340,141, filed Dec. 14, 2001, both of which are incorporated herein by reference.

### FIELD OF INVENTION

This invention relates to the field of DNA vaccines particularly for inhibiting fertility or inducing contraception.

### BACKGROUND OF THE INVENTION

The prevention of infectious diseases through use of vaccines has been realized for more than two centuries. Vaccines can be live, attenuated viruses, bacteria, inactivated organisms, toxins or partially purified preparations of organisms, polysaccharides or recombinant proteins. Other types of vaccines in development include peptides, recombinant heterologous antigens expressed by viral or bacterial vectors and plasmid DNA.

A vaccine must be effective, safe and inexpensive. Antigen presentation is critically important to the development of vaccines. Distinct immune responses are required for protection including elaboration of cytokines, neutralization by antibodies or cell-mediated immune responses.

Molecular methodologies have opened up new possibilities for vaccine production. The recent methodology, Nucleic Acid Vaccination (NAV) or DNA vaccination, has proven to be safe, effective and economic. In theory, inoculation of a plasmid DNA that encodes antigen supports in vivo expression of protein, allowing presentation of the processed protein antigen to the immune system.

The potential use of a plasmid DNA as a vaccine was first suggested in mice by the observations that administration of DNA encoding hormones or reported genes could result in expression in vivo after inoculation. Indeed, vaccination has resulted in the induction of specific antibodies and cytotoxic T lymphocytes (CTL) leading to protective efficacy such as in lethal Influenza virus (Rhodes, 1999). Since then, the use of DNA inoculation as a potential method for development of protection has been reported in different initial studies including viral (Perrin et al., 2000; Sin et al. 2000; Cherpillod et al., 2000; Osorio et al., 1999; Sixt et al., 1998; Jiang et al., 1998; Tsuti et al., FEBS Lett. 416(1): 30-34 et al., FEBS Lett. 416(1): 30-34 (1997), parasitic (Angus et al., 2000; Zhang et al., Vaccine 18(9-10): 868-874 (1999) and Stanley, Vaccine 18(9-10): 868-874 (1999); Weiss, Rev. Med. Virol. (1): 3-11 (1998), and bacterial diseases Cornell et al., J. Immunol. 163(1): 322-329 (1999) et al., Lozes et al., Vaccine (8): 830-833 (1997), Kurar and Splitter, 1997)), resulting in both cellular and humoral protective immune responses.

Nucleic acid vaccination differs from traditional vaccination. The most important is the development of a prolonged, if not, permanent immune response after a single injection of plasmid encoding protein antigen (Rhodes, 1999). Other differences include an enhanced cellular immune response, the feasibility of manipulating the NAV construct to modulate the immune response, strong immunological memory, and the unique property of not requiring the use of adjuvants.

Animal overpopulation is an ecological, economical, public health and in several countries of the world, a societal concern, particularly when 8 million dogs and cats are euthanized yearly in the U.S. alone. This problem stems mainly from the accumulation of unwanted animals through unplanned pregnancies, large litter size, ineffective contraceptive and spaying strategies and inadequate administration policies.

Traditional methods for controlling populations involve lethal methods, surgery and the administration of steroid hormone treatments. In general, the former two methods are irreversible, can be painful to the animal and are expensive. The latter requires multiple administrations over long periods and may produce undesirable side effects. An alternative procedure featuring an immunological method is desirable. Immunization or vaccination against either the female or male garnete proteins or hormones that have a key role in spermatogenesis and folliculogenesis would be advantageous. However, a zona pellucida vaccine is the only vaccine documented to be effective for contraception in mammals for the last 12 years.

The porcine zona pellucida vaccine has been proven to be effective in controlling pregnancies in domestic and wild animal species such as horses (Liu, et al. 1989), white-tailed deer (Turner, et al., 1992), tule elk (Stoops, et al., 1999), African elephants (Fayrer-Hoskins et al., 1997), and rabbits (Holland et al., Antimicrob. Agents Chemother (6): 989-991 (1985). The zona pellucida (ZP) is an egg specific protein of the mammalian oocyte involved in the binding with sperm and the induction of the acrosome reaction which is essential for the penetration and subsequent fertilization of the egg (Gupta et al., 1997; Prasad et al. 1996). Zona pellucida vaccinated animals produce antibodies as a result of an immune response that cross reacts with the zona pellucida of the vaccinated animal's oocytes preventing the interaction and penetration of the oocyte by spermatozoon hence, avoiding fertilization (Aitken et al., 1996; Paterson et al., 1996). Studies performed in mares (Liu et al., 1989) showed return to fertility in the mares when antibody titers decreased to 50% of the titers of the positive control. Hence, demonstrating the reversible effect of the vaccine. The ovarian cyclicity in these animals was not altered as demonstrated by hormonal profiles of total progesterone and normal behavioral cyclicity. Adverse effects to the vaccine have not been reported in mares, elk, deer, African elephants, bears and llamas and alpacas. However, studies in mice, monkeys, rabbits and bitches showed dysfunction of the ovary due to suppression of folliculogenesis and depletion of the pool of primordial follicles without inflammation (Paterson et al., 1996; Holland et al., Antimicrob. Agents Chemother. (6): 989-91 (1994) et al., 1994; Tung et al., 1990; Mahi-Brown et al., 1988). Other studies of native PZP inoculation in dogs (n=60), performed by this laboratory failed to demonstrate adverse effects on the ovaries of inoculated females. The present invention seeks to provide a nucleic acid vaccine based upon zona pellucida protein thereby representing a significant advancement over the prior art.

### SUMMARY OF THE INVENTION

In a first aspect, the present invention features compositions for inhibiting fertility or inducing contraception in a mammal. The compositions comprise a nucleic acid molecule encoding all or part of a zona pellucida peptide. In preferred embodiments, the peptide is a zona pellucida subunit 3, especially 3a or 3b. In especially preferred embodiments, the peptide is a porcine zona pellucida. The nucleic acid can be incorporated into an expression vector according to well known methods. This expression vector can be used to inhibit fertility or induce contraception in a mammal. This vector can also be amplified in bacterial hosts so as to allow for consistent and reliable production of the gene product under fermentation parameters and DNA purification methods.

In a second aspect, the present invention features methods for inhibiting fertility or inducing contraception in a mammal. The methods comprise the step of administering a nucleic acid molecule encoding all or part of a zona pellucida peptide. In preferred embodiments, the peptide is a zona pellucida subunit 3, especially 3a or 3b. In especially preferred embodiments, the peptide is a porcine zona pellucida. The nucleic acid can be incorporated into an expression vector according to well known methods. In some embodiments, the nucleic acid molecule is administered in a composition comprising a pharmaceutically acceptable carrier.

The present invention is also directed to the use of natural DNA derived from prokaryotes or eukaryotes, plasmid DNA, or oligonucleotides (natural or synthetic) as agents to produce transient and reversible contraception when delivered to animals or humans by injection, orally or other routes. Without wishing to be bound by theory, it is believed that transient contraception is caused by the activation of the immune system by immunostimulatory sequences (abbreviated ISS or CpG-ODN) that are known to activate the immune system (see, e.g., Rhodes, G., in Nonviral vectors for gene therapy, L. Huang, M. Hung and E. Wagner, Editors. 1999, Academic Press: San Diego.; Tighe, et al, Immunol. Today, 1998, 19 (2): 89-97; Tokunaga, et al., J. Natl. Cancer. Inst, 1984, 72 (4): 955-62; Sato, et al Science, 1996, 273 (5373): 352-4; Krieg, et al Nature, 1995, 374 (6522): 546-9 and Raz, et al Proc. Natl. Acad. Sci. USA, 1996, 93(10): 5141-5).

### BRIEF DESCRIPTION OF THE DRAWINGS

FIG. 1 describes the antibody titers observed by ELISA analysis in mice vaccinated with a single zona pellucida nucleic acid vaccine via intramuscular and intradermal administration after 3 weeks, 6 weeks and 17 weeks.

FIG. 2 describes the antibody titers observed by ELISA analysis in mice vaccinated with a single or multiple zona pellucida nucleic acid vaccinations via intradermal administration after 3 and 6 weeks.

### DESCRIPTION OF THE SPECIFIC EMBODIMENTS

In a first aspect, the present invention features compositions for inhibiting fertility or inducing contraception in a mammal. The compositions comprise a nucleic acid molecule encoding all or part of a zona pellucida peptide. In preferred embodiments, the peptide is a zona pellucida subunit 3, especially 3a or 3b. In especially preferred embodiments, the peptide is a porcine zona pellucida. The nucleic acid can be incorporated into an expression vector according to well known methods. This expression vector can be used to inhibit fertility or induce contraception in a mammal. This vector can also be amplified in bacterial hosts so as to allow for consistent and reliable production of the gene product under fermentation parameters and DNA purification methods.

In a second aspect, the present invention features methods for inhibiting fertility or inducing contraception in a mammal. The methods comprise the step of administering a nucleic acid molecule encoding all or part of a zona pellucida peptide. In preferred embodiments, the peptide is a zona pellucida subunit 3, especially 3a or 3b. In especially preferred embodiments, the peptide is a porcine zona pellucida. The nucleic acid can be incorporated into an expression vector according to well known methods. In some embodiments, the nucleic acid molecule is administered in a composition comprising a pharmaceutically acceptable carrier.

Definitions:

A “zona pellucida peptide” refers to an egg specific protein or portion thereof of the mammalian oocyte involved in the binding with sperm and the induction of the acrosome reaction which is essential for the penetration and subsequent fertilization of the egg. A “zona pellucida peptide” may refer to the naturally occurring protein or a portion thereof, or to muteins, mutants or fragments thereof. Such a peptide may occur in or be taken from any mammalian species.

An “immunogen” refers to a peptide, polypeptide or protein which is “immunogenic,” i.e., capable of eliciting an immune response, in this case against zona pellucida antigens. An immunogenic composition of the invention can be a composition comprising the polypeptide or a recombinant vector which encodes the polypeptide.

In addition, the precise sequence of the nucleic acid molecules of the invention need not be identical and may be “substantially identical” to a sequence disclosed here. As explained below, these variants are specifically covered by the term zona pellucida peptide or a nucleic acid molecule encoding a zona pellucida peptide.

In the case where the polynucleotide sequence is transcribed and translated to produce a functional polypeptide, one of ordinary skill will recognize that because of codon 5 degeneracy a number of polynucleotide sequences will encode the same polypeptide. These variants are specifically covered by the above term. In addition, the term specifically includes those sequences substantially identical (determined as described below) with a sequence disclosed here and that encode proteins that are capable of inducing immune response against zona pellucida antigens.

Two nucleic acid sequences or polypeptides are said to be “identical” if the sequence of nucleotides or amino acid residues, respectively, in the two sequences is the same when aligned for maximum correspondence as described below. The term “complementary to” is used herein to mean that the complementary sequence is identical to all or a portion of a reference polynucleotide sequence.

Optimal alignment of sequences for comparison can use any means to analyze sequence identity (homology) known in the art, e.g., by the progressive alignment method of termed “PILEUP” (see below); by the local homology algorithm of Smith & Waterman, Adv. Appl. Math. 2: 482 (1981); by the homology alignment algorithm of Needleman & Wunsch, J. Mol. Biol., 48:443 (1970); by the search for similarity method of Pearson (1988) Proc. Natl. Acad. Sci. USA 85: 2444; by computerized implementations of these algorithms (GAP, BESTFIT, FASTA, and TFASTA in the Wisconsin Genetics Software Package, Genetics Computer Group, 575 Science Dr., Madison, Wis.); ClustalW (CLUSTAL in the PC/Gene program by Intelligenetics, Mountain View, Calif., described by Higgins (1988) Gene, 73:237-244; Corpet (1988) Nucleic Acids Res. 16:10881-90; Huang (1992) Computer Applications in the Biosciences 8:155-65, and Pearson (1994) Methods in Molec. Biol. 24:307-3 1), TreeAlign, MALIGN, and SAM sequence alignment computer programs; or, by inspection. See also Morrison (1997) Mol. Biol. Evol. 14:428-441, as an example of the use of PILEUP. PILEUP, creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35:351-360 (1987). The method used is similar to the method described by Higgins & Sharp (1989) CABIOS 5:151 153. The program can align up to 300 sequences of a maximum length of 5,000. The multiple alignment procedure begins with the pairwise alignment of the two most similar sequences, producing a cluster of two aligned sequences. This cluster can then be aligned to the next most related sequence or cluster of aligned sequences. Two clusters of sequences can be aligned by a simple extension of the pairwise alignment of two individual sequences. The final alignment is achieved by a series of progressive, pairwise alignments. The program can also be used to plot a dendogram or tree representation of clustering relationships. The program is run by designating specific sequences and their amino acid or nucleotide coordinates for regions of sequence comparison.

Another example of an algorithm that is suitable for determining sequence similarity is the BLAST algorithm, which is described in Altschul (1990) J. Mol. Biol. 215:403-410. Software for performing BLAST analyses is publicly available through the National Center for Biotechnology Information, http://www.ncbi.nlm.nih.gov/; see also Zhang et al., Vaccine 18(9-10): 868-874 (1997), Genome Res. 7:649-656 (1997) for the “PowerBLAST” variation. This algorithm involves first identifying high scoring sequence pairs (HSPs) by identifying short words of length W in the query sequence that either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are extended in both directions along each sequence for as far as the cumulative alignment score can be increased. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLAST program uses as defaults a wordlength (W) of 11, the BLOSUM62 scoring matrix (see Henikoff (1989) Proc. Natl. Acad. Sci. USA 89:10915) alignments (B) of 50, expectation (E) of 10, M=5, N=−4, and a comparison of both strands. The BLAST algorithm performs a statistical analysis of the similarity between two sequences (see, e.g., Karlin (1993) Proc. Natl. Acad. Sci USA 90:5873-5787). One measure of similarity provided by the BLAST algorithm is the smallest sum probability (P(N)), which provides an indication of the probability by which a match between two nucleotide or amino acid sequences would occur by chance.

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” of polynucleotide sequences means that a polynucleotide comprises a sequence that has at least 75% sequence identity, preferably at least 85%, more preferably at least 90% and most preferably at least 95%, compared to a reference sequence using the programs described above using standard parameters. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of at least 40%, preferably at least 60%, more preferably at least 90%, and most preferably at least 95%. Peptides or polypeptides that are “substantially similar” share sequences as noted above except that residue positions which are not identical may differ by conservative amino acid changes. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, aspartic acid-glutamic acid, and asparagine-glutamine.

Another indication that nucleotide sequences are substantially identical is if two molecules hybridize to each other, or a third nucleic acid, under stringent conditions. Stringent conditions are sequence dependent and will be different in different circumstances. Generally, stringent conditions are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Typically, stringent conditions will be those in which the salt concentration is about 1 molar at pH 7 and the temperature is at least about 60° C.

“Conservatively modified variations” of a particular nucleic acid sequence refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given polypeptide. For instance, the codons CGU, CGC, CGA, COG, AGA, and AGG all encode the amino acid arginine. Thus, at every position where an arginine is specified by a codon, the codon can be altered to any of the corresponding codons described without altering the encoded polypeptide. Such nucleic acid variations are “silent variations,” which are one species of “conservatively modified variations.” Every nucleic acid sequence herein that encodes a peptide or polypeptide also describes every possible silent variation. One of ordinary skill will recognize that each codon in a nucleic acid (except AUG, which is ordinarily the only codon for methionine) can be modified to yield a functionally identical molecule by standard techniques. Accordingly, each “silent variation” of a nucleic acid that encodes a peptide or polypeptide is implicit in each described sequence.

The term “conservatively modified variations” refers to individual substitutions, deletions or additions which alter, add or delete a single amino acid or a small percentage of amino acids (typically less than 5%, more typically less than 1%) in an encoded sequence, where the alterations result in the substitution of an amino acid with a chemically similar amino acid; and the alterations, deletions or additions do not alter the structure, function and/or immunogenicity of the sequence. Conservative substitution tables providing functionally similar amino acids are well known in the art. The following six groups each contain amino acids that are conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

The term “inhibiting fertility” means reducing the number of eggs fertilized by sperm or reducing the percentage of females who conceive offspring in a population. Similarly, the term “inducing contraception” means decreasing the incidence of fertilization in a population.

Zona Pellucida Nucleic Acid

The present invention uses a zona pellucida based nucleic acid vaccine for population control starting with the mouse (Mus musculus) model and Porcine Zona Pellucida 3-alpha (PZP-3a)(Genbank LI 1000) (SEQ. ID NO:1) or Porcine Zona Pelluicda 3-beta (PZP-3b) (Genbank L22169) (SEQ. ID NO: 2) as the encoded protein antigen.

Zona pellucida (ZP) DNA is well conserved in all mammalian species, such as mouse, chicken, pig, cow, dog, cat, marsupials, non-human primates and human (Genbank database). Hence, the present invention uses the contraceptive/sterilant effectiveness in heterologous (eg: porcine zona pellucida, PZP 3-a in mice) as well as in homologous (eg: canine zona pellucida, CZP-2 and CZP-3 in dogs) mammalian systems.

The present invention encompasses other nucleic acid vaccine constructs that include the porcine ZP-3 beta (L22169); Canine Zona Pellucida-3 (E06068), Canine Zona Pellucida-2 (E07830); Mouse Zona Pellucida-3 (M20026), Mouse Zona Pellucida-2 (NM 011775) and in the first stages for the Canine Zona-A (U05779); Feline Zona Pellucida-3 or FZP-3 (E06599, E06596), FZP-2 (E07930, D45067), FZP-A (U05776), FZP-B (U05777), FZP-C (U05778); Mouse Zona Pellucida-1 (NM 009580) and Monkey Zona Pellucida-3 or MZP-3 (X82639), MZP-2 (Y10690), MZP-1 (Y10381, Y10382, Y10383) and Human Zona Pellucida or HZP (BC005223), HZP-2 (XM007848, NM003460, M90366), HZP-A (XM032143, NM007155), HZP-B (U05781) and HZP-4 (NM021186), all of which sequences are herein incorporated by reference. In other embodiments, the invention features constructs in which epitopes of zona pellucida, specific for the immune-mediated function, are conjugated or linked with major DNA constructs.

The present invention relates to immunogenic compositions capable of eliciting an immunogenic response directed to a zona pellucida peptide. This can be accomplished by administering either the nucleic acids disclosed herein or polynucleotides encoding polypeptides having substantial identity. The encoded polypeptides can be readily designed and manufactured utilizing various recombinant DNA or synthetic techniques well known to those skilled in the art. For example, the polypeptides can vary from the naturally-occurring sequence at the primary structure level by amino acid, insertions, substitutions, deletions, and the like. These modifications can be used in a number of combinations to produce the final modified protein chain. For instance, fusion proteins comprising the polypeptides of the invention fused to various heterologous proteins can be prepared.

The amino acid sequence variants can be prepared with various objectives in mind, including facilitating purification and preparation of the recombinant polypeptide. The modified polypeptides are also useful for modifying plasma half life, improving therapeutic efficacy, and lessening the severity or occurrence of side effects during therapeutic use. The amino acid sequence variants are usually predetermined variants not found in nature but exhibit the same immunogenic activity as naturally occurring zona pellucida polypeptides. The nucleotide sequences can be modified according to standard techniques to yield the desired polypeptides, fusion proteins, or fragments thereof, with a variety of desired properties.

Nucleic Acid Vaccine

Nucleic Acid Vaccination (NAV) consists of the inoculation of a nonreplicating expression vector or plasmid (DNA) that supports in vivo expression of an encoded protein allowing presentation of the processed protein antigen to the immune system. This was first demonstrated in mice after inoculation of DNA encoding hormones or reported genes (Rhodes, 1999). The use of NAV for the development of both cellular and humoral immune responses have been reported in viral diseases produced for rabies, herpes simplex, distemper, parvovirus and HIV (Perrin et al., 2000; Sin et al., 2000; Cherpillod et al., 2000; Osorio et al., 1999; Sixt et al., 1998; Jiang et al., 1998; Tsuti et al., FEBS Lett. 416(1): 30-34 (1997), bacteria such as Salmonella and Mycobacterium (Cornell et al., J. Immunol. 163(1): 322-329 (1999) et al., 1999; Lozes et al., Vaccine (8): 830-833 (1997) et al., 1997 Kurar and Splitter, 1997) and parasitic diseases such as Toxoplasmosis and Malaria (Angus et al., 2000; Zhang et al., Vaccine 18(9-10): 868-874 and Stanley, 1999; Weiss, Rev. Med. Virol. (1): 3-11 (1998).

The present invention demonstrates successfully administering ZP-NAV, especially PZP-3 alpha-NAV, to achieve infertility in female mice. The ZP 3a-DNA vaccine continuously expresses the antigen in the individual's cells, producing a constant immune response due to the development of immunological memory and/or repriming of the immunity due to permanent exposure to the antigen. The resulting immunological response prevents fertilization of the inoculated female animal. As noted above, the nucleic acid need not encode a protein antigen. In some embodiments, the administered nucleic acid is plasmid DNA or a natural or synthetic ISS oligonucleotides. ISS are single stranded oligonucleotides of 6 to 40 bases built around a central CpG dinucleotide sequence. The central CG dinucleotide is required for immune stimulation and oligonucleotides lacking this feature do not activate the immune system. Active ISS are present in all bacterial derived plasmid DNA. They can also be made synthetically as single or double stranded oligonucleotides of about 5 to about 100 bases. Typically, oligonucleotides of the invention are between about 10 and about 50 nucleotides in length, between about 15 and about 25 nucleotides, or between about 20 and about 30 nucleotides. In many embodiments they are about 40 nucleotides in length.

Methods for preparing synthetic oligonucleotides are well known to those of skill in the art. A “synthetic” oligonucleotide refers to a polynucleotide synthesized using in vitro chemical methods, e.g., by using a machine that synthesizes polynucleotides using the phosphodiester method, the diethylphosphoramidite method, the phosphotriester methods, the solid support method, and other methods known to those skilled in the art.

Techniques for rendering oligonucleotides nuclease-resistant include those described in PCT Publication WO 94/12633. A wide variety of useful modified oligonucleotides may be produced, including oligonucleotides having a peptide-nucleic acid (PNA) backbone (Nielsen et al., 1991, Science 254:1497) or incorporating 2′-O-methyl ribonucleotides, phosphorothioate nucleotides, methyl phosphonate nucleotides, phosphotriester nucleotides, phosphorothioate nucleotides, and phosphoramidates.

Nucleic Acid Vaccine Compositions

The nucleic acids of the present invention can be used in pharmaceutical and vaccine compositions that are useful for administration to mammals, particularly dogs and cats, to inhibit fertility or induce contraception. The compositions are also useful in humans, particularly when using immunostimulatory sequences for transient contraception The compositions are suitable for single administrations or a series of administrations. When given as a series, inoculations subsequent to the initial administration are given to boost the immune response and are typically referred to as booster inoculations.

Thus, the invention provides compositions for parenteral administration that comprise a solution of the nucleic acids of the invention dissolved or suspended in an acceptable carrier, preferably an aqueous carrier. A variety of aqueous carriers may be used, e.g., water, buffered water, 0.4% saline, 0.3% glycine, hyaluronic acid and the like. These compositions may be sterilized by conventional, well known sterilization techniques, or may be sterile filtered. The resulting aqueous solutions may be packaged for use as is, or lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc.

For solid compositions, conventional nontoxic solid carriers may be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95% of active ingredient and more preferably at a concentration of 25%-75%.

The vaccines of the invention contain as an active ingredient an immunogenically effective amount of the nucleic acids as described herein. Useful carriers are well known in the art, and include, e.g., thyroglobulin, albumins such as human serum albumin, tetanus toxoid, polyamino acids such as poly(D-lysine:D-glutamic acid), influenza, hepatitis B virus core protein, hepatitis B virus recombinant vaccine and the like. The vaccines can also contain a physiologically tolerable (acceptable) diluent such as water, phosphate buffered saline, or saline, and further typically include an adjuvant. Adjuvants such as quill-A, cholesterol, aluminum phosphate, aluminum hydroxide, or alum are materials well known in the art.

Administration

The pharmaceutical compositions of the invention are intended for parenteral, topical, oral or local administration. Preferably, the pharmaceutical compositions are administered parenterally, e.g., intravenously, subcutaneously, intradermally, or intramuscularly.

The nucleic acids of the present invention are useful as vaccines, for inhibiting fertility or inducing contraception. Compositions containing the nucleic acids are administered to a subject, giving rise to an immune response against the peptides encoded thereby. An amount of a composition sufficient to result in this inhibition is defined to be an “immunologically effective dose.” In this use, the precise amounts will depend on the patient's state of health and weight, the mode of administration, and the nature of the formulation. Typically, an immunologically effective dose for the nucleic acids the dose will be between about 5 to about 500 .mg/kg body weight, preferably about 50 to about 100 mg/kg body weight.

Administration of DNA is described, for instance, in Wolff et al., Science 247: 1465-1468 (1990), as well as U.S. Pat. Nos. 5,580,859 and 5,589,466. Several pharmaceutical or prophylactic formulations can be utilized with the purified plasmid DNA of this invention. Plasmid DNA can be prepared in solution or in desiccated form by several methods known to those skilled in the art. More specifically, the sample can be diluted to the desired buffer or DNA concentration directly and packaged directly into receptacles for administration or storage. The sample may also be dialyzed against the desired solution or diluent to be used to administer the product. The purified DNA can be removed from the elution buffer or other solutions by a variety of techniques known to those skilled in the art; typically lyophilization or precipitation of polynucleotide salts in solvents are utilized. In this invention the preferred method is to avoid the use of solvents or other hazardous chemicals to precipitate the DNA. PEG 8000 is the preferred method and is used in a similar manner described above. DNA can then be resuspended in a diluent or other formulation of choice. Although a number of diluents and pharmaceutical/prophylactic formulations are known to those skilled in the art, the diluent is most likely to be high quality water containing physiological concentrations of salt or carbohydrates to remove stinging or burning sensation when administered by standard methods which require penetration of the outer skin surface.

DNA can be stored in suspension or as a salt of PEG pellet at several temperatures ranging from −80° C. to 25° C. Most preferably DNA is resuspended in sterile diluent containing 0.15 M NaCl and 25 mM phosphate buffer pH 7.2.

Although plasmid DNA can be delivered subcutaneously, intramuscular, intraperitoneally, intradermally, intranasally, orally or topologically; the preferred method is by intramuscular injection. The purified DNA can be administered in a volume of 0.05 ml to 2 ml of a physiological sterile saline containing between 0.1 μg to 1 mg of DNA. Most preferably the volume is 0.5 ml-1ml containing 100-500 μg of plasmid DNA.

The isolated nucleic acid sequences coding for desired polypeptides can also be used to transform viruses that transfect host cells in the susceptible organism. Live attenuated viruses, such as vaccinia viruses are convenient alternatives for vaccines because they are inexpensive to produce and are easily transported and administered. Vaccinia vectors and methods useful in immunization protocols are described in, e.g. U.S. Pat. No. 4,722,848. Other suitable vectors include, but are not limited to, pox viruses, such as, canarypox and cowpox viruses, and other animal viruses.

The present invention is described below. Although these examples describe broadly the methods used, they are not intended to be limited by these examples.

### EXAMPLE 1

Material and Methods

Animals: 6 to 8 week old female and 14 week old male Balb/c mice were obtained from Animal Resources Services at the University of California at Davis. Mice are housed consistent with the guidelines of the Laboratory Animal Use Protocol.

Immunization protocol: Five groups of female Balb/c mice were organized (see table # 1). Group #1 received 25 μg of pND+PZP-3a NAV by a single intradermal (ID) injection at the base of the tail. Group #2 was injected with 50 μg of PND+PZP-3a via intramuscular (IM) injection into the hindquarters (quadriceps femoralis). Group #3 received only pND+NP (vector). Group #4 was injected with phosphate buffer saline (PBS) via ID and group #5 was vaccinated via ID with 130 μg of whole porcine zona pellucida (PZP) protein in Freund's Complete Adjuvant and received a booster of 130 μg PZP in Freund's Incomplete 3 weeks later. DNA plasmids were dissolved in NaCl, 0.1 5M in a final volume of 100 μl per dose. Blood samples were taken at 3, 6 and 17 weeks post injection by retroorbital bleeding.

TABLE #1
Mice Groups and Immunization Protocol

| Group | # animals | Via | Antigen | Doses (μg) |
|---|---|---|---|---|
| 1 | 5 | ID | pND + PZP-3a | 25 |
| 2 | 5 | IM | pND + PZP-3a | 50 |
| 3 | 3 | ID | pND + NP | 25 |
| | 2 | IM | pND + NP | 50 |
| 4 | 5 | ID | PBS | 100 ul |
| 5* | 5 | ID | crude PZP protein | |

*Mice receive a booster of 130 μg crude PZP protein in Freund's Incomplete adjuvant.

Mating challenge: 4 adult males, 16 weeks of age, were used for mating of the females: 1 male/2-3 females remained in a cage together for 6 consecutive days between weeks 8-9 and again on week 18 post injection. Results are shown in table # 2.

TABLE #2
Mating Challenge Results from Mice (BABL/c)
Injected with Single ZP-NAV

| # Mice/ | | | 9 week | | 17 week | |
|---|---|---|---|---|---|---|
| group | Antigen | Via | # Pregnant | # pups | # Pregnant | # pups |
| 5 | ZP + ND | ID | 1/5 | 3 | 1/5 | 5 |
| 5* | ZP + ND | IM | 2/5 | 13 | 2/5 | 11 |
| 5** | ND + NP | ID, IM | 2/5 | 11 | 3/3 | 23 |
| 3* | PBS | ID | 3/3 | 3/3 | 2/2 | 14 |

*One mouse died on 16 week
**Two mice death on 16 week
\hat{ } Same mice that got pregnant on 9 week

Plasmid construction: Porcine Zona Pellucida-3 alpha (PZP-3a) sequence was reported by Yurewicz et al., 1993. The sequence was obtained from GenBank database (L11000).

PZP-3a was cloned from total RNA from pig ovaries by RT-PCR (Reverse Transcriptase Polymerase Chain Reaction). Fresh pig ovaries were collected in RNA later (Ambion, Cat. No. 7021) RNA stabilization solution and stored at 4° C. until total RNA was isolated using a RNA/DNA Midi kit (Quiagen Cat.No. 14142) following manufacturers instructions. To prepare the template, 1.5 μg of total RNA was reverse transcribed in a final 12 μl reaction using 30 pmol of the following custom oligo RT primer (Gibco, Life Technologies Inc.):

5′ 3′

GGTTTTATTGACACATTTG

heated at 70° C. for 10 minutes and chilled on ice for 2 minutes. Afterwards, 5× buffer (Gibco, Life Technologies Inc.Cat.No. 28025-013), 10 mM dNTP mix (Perkin Elmer), 0.1 M DTT (Gibco, Life Technologies Inc.Cat.No. 15508-013) and 40 U of RNAsin (Promega Cat.No. N2111) were added. The reaction was incubated for 2 minutes at 42° C. and 1 μl of M-MLV (Moloney Murine Leukemia Virus) reverse transcriptase enzyme (Gibco, Life Technologies Inc. Cat. No. 28025-013) was added. Incubations were followed at 25° C. for 10 minutes, 42° C. for 50 minutes and 70° C. for 15 minutes. PCR amplification was performed in a 50 μl reaction (nuclease free water, 5 μl of 10×Pfu buffer and 20 μl of 10 mM dNTPs) using 1-3 μl of pig ovary template, 0.5 μl of Pfu polymerase (Perkin Elmer, Roche) and 20 pmol of each of the following customized primers (Gibco, Life Technologies Inc.):

Upper primer 5′ 3′
CCGGTACCTCTCCGCAGGCGCTATG
Lower primer 5′ 3′
CCGTCGACGTCTGAGTAACTCATCTC

Reactions were cycled at 95° C. for 1 mm, 95° C. for 45 sec, 55° C. for 45 sec, 72° C. for 5 min and 72° C. for 5 min for 30 cycles using a capillary thermal cycler. The resulting clone PZP-3a was amplified again using the Zero Blunt TOPO PCR cloning kit (Invitrogen Cat. No K2800-20). The cloning reaction was set as follows:
PCR product (cPZP-3a) 1 μl
Salt solution (provided with the kit) 1 μl
Sterile water 3 μl
TOPO vector (provided with the kit) 1 μl
Total volume 6 μl

The cloning reaction was mixed gently for 5 minutes at room temperature and immediately used for transformation into chemically competent E. coli cells DH5-alpha (Gibco, Life Technologies, Inc. Cat.No. 18258-012). Briefly, 2 μl from this reaction was added into a vial containing 100 μl of chemically competent E. coli (DH5-a) and flicked gently, followed by incubation on ice for 30 minutes, heat shocked for 20 seconds at 42° C. without agitation and immediately transferred into ice for 2 min. 100 μl of LB prewarmed at 37° C. was added. The vial was placed in an orbital shaker at 37° C. with horizontal agitation (225 rpm) for 1 h. Finally, 10 μl and 90 μl from the transformed bacteria were spread on a prewarmed selective plate (LB medium+50 μg/ml Kanamycin) and incubated overnight at 37° C. The following day, 10 colonies were selected for analysis of positive clones. Colonies were selected and cultured in 3 ml of LB medium+50 μg/ml Kanamycin overnight at 37° C. in an orbital shaker (225 rpm). The next day, miniplasmid preps were prepared to isolate the plasmid DNA from the bacterial cultures by removing 1.5 ml from each overnight culture and placed in a microcentrifuge vial, spun at 14,000 rpm at room temperature for 2 minutes, resuspending the pellet in 60 μl GTE (1M Tris pH7, 0.5 M EDTA pH 8 and 20% glucose) and 40 μl of 10 mg/ml lysozyme in GTE followed by a 5 minute incubation at room temperature. 200 μl of 0.2 M NaOH 1% SDS was added and incubated on ice for 10 min, addition of 150 μl SM KOAc (ice cold) pH 4.8, incubation on ice for 10 min and centrifuged at 14,000 rpm for 10 min at 4° C. The supernatant was transferred to a new tube. For extraction, 200 μl of phenol and 200 μl of chloroform was added to the supernatant, and the mixture was vortexed and centrifuged at 14,000 rpm for 5 minutes at room temperature. 450 μl of the top phase was removed (DNA) to a new vial and 900 μl of cold ethanol was added. The mix was vortexed for 5 sec and incubated on ice for 5 min. To pellet the plasmid DNA, a final centrifugation at 14,000 rpm for 15 minutes at 4° C. was performed. The DNA pellet was air dried for 7 min and resuspended in 50 μl nuclease free (Promerga Cat.No. DP 1193) water with 1 μl 10 mg/ml RNase A (AMRESCO Cat.No. 0675). Vials were labelled and stored at −20° C. One culture of miniplasmid prep (plasmid DNA) showing the correct fragment sizes of DNA after 1 h digestion at 37° C. with Eco RI (New England, Biolabs, Cat.No. #101S) was selected and replated in a petri culture plate. In order to recover the PZP-3a clone from the plasmid DNA, 20 μl of this miniprep was digested overnight at 37° C. with EcoRI nuclease followed by agarose gel DNA isolation with Geneclean II (BIS 101, Cat. No 1001-400). After overnight digestion, the whole reaction was loaded in a 0.8% TBE agarose gel. Band was excised from the gel with a sterile bisturi and placed in a sterile microcentrifuge vial. Half volume (from the cut gel) of TBE modifier and 4.5 volumes of NaI was added and incubated at 55° C. to melt the gel for 10-15 mm until it was dissolved. 5 μl of “glassmilk” suspension was added to the mixture and vortexed every 2 minutes for a 10 minute period to keep it in suspension and centrifuged at 13,000 rpm for 1 min at room temperature. Supernatant was discarded and pellet was washed 3 times with 500 μl of “New wash solution”. In each wash the pellet was resuspended repeatedly with the “New wash solution” and centrifuged at 13,000 rpm for 1 minute. The pellet was eluted in 20 μl, sterile TE and centrifuged at 13,000 rpm for 1 minute. The supernatant was placed in a new vial and recentrifuged again. Supernatant containing our PZP-3a clone was transferred to a new vial and now it is called the “Insert”.

Similarly, Vector (pND) was also digested overnight at 37° C. with EcoRI restriction enzyme and treated with 2 μl CIAP (Gibco, Life Technologies Inc.Cat.No. 18009-027) for 15 minutes at 37° C., 1 μl of 5 mM EDTA followed by 20 min incubation at 65° C. and isolated by Geneclean II agarose gel isolation kit as described before.

A TBE agarose gel electrophoresis was performed at 0.8% to measure the concentration of insert (cPZP-3a) and vector (pND) and to facilitate the use of correct quantities of insert and vector required for ligation.

Ligation Reaction:

| | |
|---|---|
| Insert (cPZP-3a) | 0.7 μl (35 μg) |
| Vector (pND) | 7.6 μl (91.2 μg) |
| Buffer 10× | 1.0 μl |
| T4 DNA ligase | 1.0 μl |
| Total volume | 10 μl |

Ligation reaction was performed overnight at 16° C.

Transformation of the new plasmid (pND+PZP-3a) in E. coli (DH5-a) and growth in 25 ml LB media+ampicillin (1:2000) culture media for Midi preps (Quiagen, Cat.No ______) was prepared and processed following manufacture's instructions.

Digestions with Pst I and Hinc II (New England, Biolabs, Cat.No 140S and 103S) restriction enzymes and sequencing were used to screen and confirm the new plasmid. The gene encoding the porcine zona pellucida 3-a (PZP-3a) was successfully inserted into pND vector.

Transfection of hamster fibroblasts kidney cells (hfkc): HKC cells (ATCC Cat.No ______) were grown to 50% confluence at 37° C. in a humidified 5% CO₂ atmosphere in 35 mm wells in Dulbecco's Modified Eagle Medium (Gibco, Life Technologies Inc. Cat.No.11965-092) containing 100 U/mL each penicillin and streptomycin and 10% fetal calf serum and were transfected with 5 μg pND+PZP-3a plasmid DNA from midiplasmid prep with 25 μl of Geneporter (Gene Therapy Systems Inc.Cat. No T201007). After 2 days, medium was collected, cell monolayers were washed twice with 2 ml of PBS and then scraped into 1 ml of DEM. Each transfection was placed in a microcentrifuge vial and disrupted by pipeting. Finally, vials containing samples were boiled for 4 min and stored at −20° C. until used for western blot.

Western blot Analysis: After SDS-PAGE electrophoresis, polypeptides were transferred onto PDVF Immobilon-P transfer membrane (Millipore Cat.No. IPVH00010. Dog anti PZP serum (1:500) diluted in PM (3% Non fat powder milk in BBS 1×) was incubated with the membrane overnight at 4° C.

Hereafter, all washes and incubations were performed at room temperature. Membrane was rinsed with BBS 1× and washed with PM for 10 minutes at room temperature followed by an incubation of 2 hours with 1:2000 alkaline-phosphatase-conjugated affinipure rabbit anti-dog IgG antibody (Jackson Immunoresearch Laboratories, Cat. No. 304-055-003). The membrane was rinsed with BBS 1× and washed with PM for 10 minutes. Finally, the developer 100 μl BCIP (Fisher Biotech, Fisher Scientific Cat.No.BP1610-100)+50 μl NBT (Fisher Biotech, Fisher Scientific Cat.No BP108-1)+15 ml of APP buffer was added.

Sera from vaccinated female mice with NAV-PZP-3a will be analyzed by western blot using this same method.

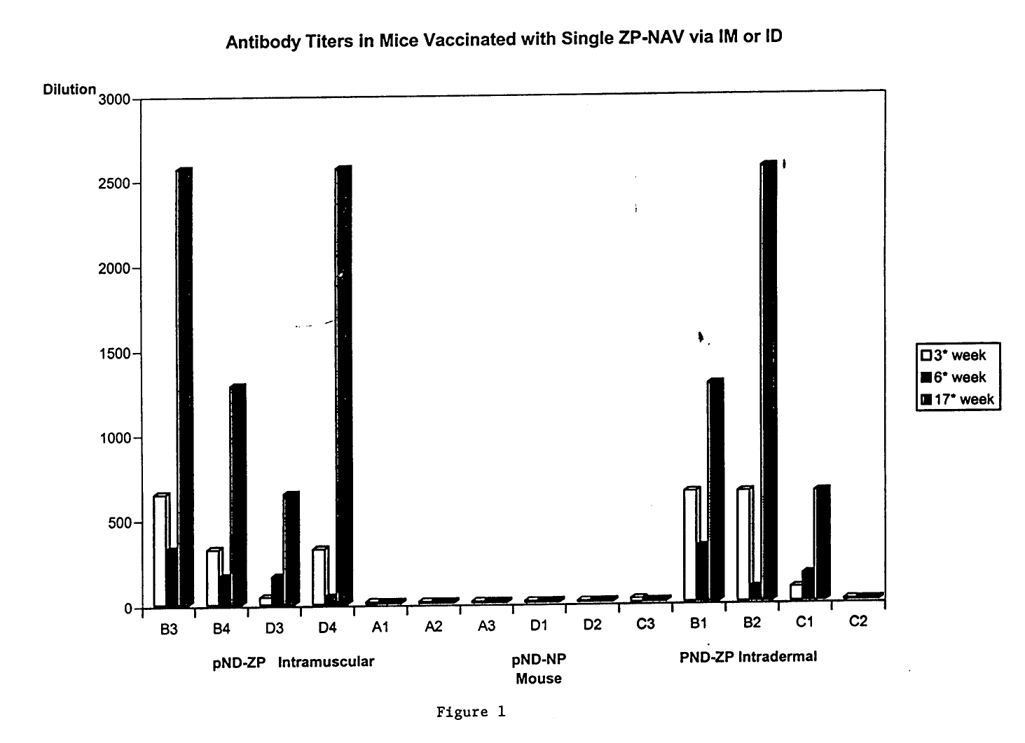

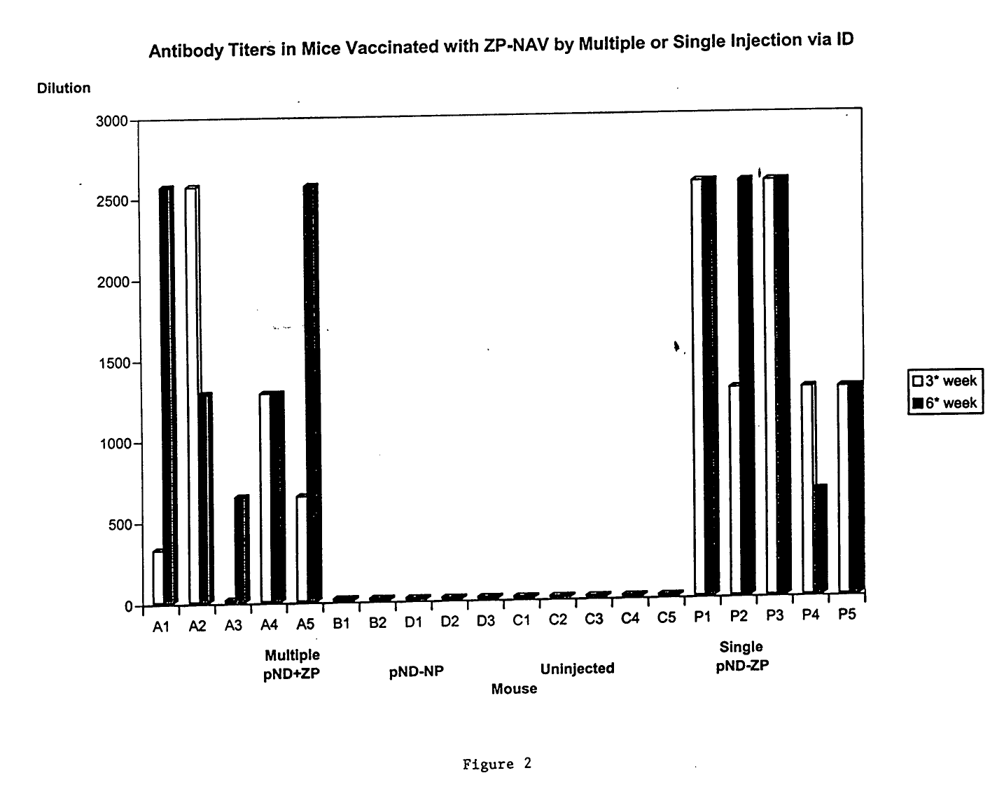

ELISA: The ELISA assay was performed to measure PZP-3a antibodies levels (FIGS. 1 and 2) and isotyping assays to evaluate the predominant type of immune response (cellular/humoral or Th1/Th2) (see Table # 3).

Briefly, 50 μl of a 10 μg/ml of crude PZP antigen in solution with BBS 1× buffer was placed in each well of a flat bottom multi well micro-ELISA plate (Costar, Cat.No.3690) and incubated overnight at 4° C. Followed by 6 washes with 150 μl of washing solution (BBS 1×+0.05% Tween) and incubation for 2 hours with 50 μl of: blocking solution (BBS 1×+1% bovine serum albumin) for nonspecific binding sites. 2) Overnight incubation at 4° C. with 50 μl of sample test serum in serial dilutions from 1/20 to 1/2580. 3) Two hour incubation with 50 μl of alkaline-phosphate/biotnylated goat anti mouse diluted in BBS 1×1:2000) Finally, 50 μl substrate solution of 1 mg p-nitro phenyl phosphate/ml in carbonate buffer pH 8.4 supplemented with 1 μl of MgCl₂/ml was added to each well and scanned at 410 wave length for absorbance with a micro-ELISA auto reader.

The anticipated removal of spleens from treated and control mice will be used for evaluation of the cellular immune response by ELISPOT, T-cell proliferation assay and cytokine measurements by flow cytometry or ELISA assay.

TABLE #3
ELISA Isotyping Assay in Female Mice Injected with
ZP-NAV and Standard ZP Vaccine
Ig G Isotype (O.D)

| ID # | Via | Antigen | 1 | 2a | IgG 1/IgG 2a | Response |
|---|---|---|---|---|---|---|
| B1 | ID | pND-ZP | 0.280 | 0.329 | 0.85 | Th 1 |
| C1 | ID | pND-ZP | 0.402 | 0.151 | 2.66 | Th2 |
| C1* | ID | pND-ZP | 0.293 | 0.359 | 0.81 | Th1 |
| B1 | ID | pND-ZP | 0.735 | 1.226 | 0.60 | Th1 |
| D4* | IM | pND-ZP | 0.278 | 0.746 | 0.37 | Th1 |
| B2* | IM | pND-ZP | 0.237 | 0.655 | 0.36 | Th1 |
| B4* | IM | pND-ZP | 0.299 | 0.608 | 0.49 | Th1 |
| D3* | IM | pND-ZP | 0.180 | 0.733 | 0.25 | Th1 |
| B2 | IM | pND-ZP | 0.231 | 0.952 | 0.24 | Th1 |
| B3 | IM | pND-ZP | 0.265 | 0.944 | 0.28 | Th1 |
| D3 | IM | pND-ZP | 0.223 | 0.984 | 0.23 | Th1 |
| B4 | IM | pND-ZP | 0.212 | 1.106 | 0.19 | Th1 |
| W1 | ID | ZP protein | 0.452 | 0.096 | 4.72 | Th2 |
| W2 | ID | ZP protein | 0.460 | 0.107 | 4.30 | Th2 |
| W3 | ID | ZP protein | 0.414 | 0.137 | 3.02 | Th2 |
| W4 | ID | ZP protein | 0.446 | 0.103 | 4.33 | Th2 |
| A1 | ID | ND + NP | 0.071 | 0.072 | | |
| A3 | ID | ND + NP | 0.072 | 0.077 | | |

*Blood sample taken at 3 weeks after injection. The remaining samples were taken at week 6.

TABLE #4
Antibody Titers and Outcome for Female
Mice Injected Once with ZP-NAV
Part I

| | | | | 6* week | |
|---|---|---|---|---|---|
| Animal ID | Antigen | Via | 3* week | Preg | 17* week Preg |
| B1 | PZP + ND | ID | 1/640 | 1/320− | 1/1280− |
| B2 | PZP + ND | ID | 1/640 | 1/80− | 1/2560− |
| C1 | PZP + ND | ID | 1/80 | 1/160− | 1/640− |
| C2 | PZP + ND | ID | <1/20 | <1/20− | <1/20− |
| C3 | PZP + ND | ID | 1/20 | <1/20+ | <1/20+ |
| B3 | PZP + ND | IM | 1/640 | 1/320+ | 1/2560+ |
| B4 | PZP + ND | IM | 1/320 | 1/160− | 1/1280 n.a (dead) |
| D3 | PZP + ND | IM | 1/40 | 1/160− | 1/640+ |
| D4 | PZP + ND | IM | 1/320 | 1/40+ | 1/2560− |
| D1 | ND + NP | IM | <1/20 | <1/20− | <1/20+ |
| D2 | ND + NP | IM | <1/20 | <1/20+ | <1/20 N.A (dead) |
| A1 | ND + NP | ID | <1/20 | <1/20− | <1/20+ |
| A2 | ND + NP | ID | <1/20 | <1/20+ | <1/20+ |
| A4 | ND + NP | ID | <1/20 | <1/20− | <1/20 n.a (dead) |
| W1 | PBS | ID | <1/20 | <1/20+ | <1/20+ |
| W2 | PBS | ID | <1/20 | <1/20+ | <1/20+ |
| W3 | PBS | ID | <1/20 | <1/20+ | <1/20 n.a (dead) |

Part II

| | | | 2* week | 5* week | 9* week Preg. |
|---|---|---|---|---|---|
| W1 | PZP protein | ID | >1/2560 | >1/2560 | >1/2560− |
| W2 | PZP protein | ID | >1/2560 | >1/2560 | >1/2560− |
| W3 | PZP protein | ID | >1/2560 | >1/2560 | >1/2560− |
| W4 | PZP protein | ID | >1/2560 | >1/2560 | >1/2560− |

For part I, mice were challenged to mating during week 9* (for 5 consecutive days) and week 18* (for 7 consecutive days).
For part II, mice were mated during week 11*. These female received an initial injection of regular ZP and a booster after 3 weeks.

Ovarian Cyclicity: To evaluate ovarian cyclicity vaginal smears were performed using Quik Dip (Mercedes Medical Supplies Cat.No.320A).

T-cell Proliferation Assay: Spleens will be aseptically removed from mice and single cell preparations will be made as described (Gazzinelli et al., 1991). Cells (2×10⁵/well) will be plated in RPMI with 10% fetal calf serum onto 96 well microtiter plates. One microgram of purified PZP-3a will be added to each well and cultures will be maintained in 5% CO₂ for 48 h. Cytokines released into the medium will be quantified by ELISA (R&D Systems, Minneapolis). Data will be expressed as means±SD.

Indirect Immunofluorescence: Oocytes from untreated female mice will be recovered as described (Lorenzo, 1992). The recovered oocytes will be washed in 0.1M PBS (pH 7.4) and then incubated for 30 minutes with serum dilutions (ELISA positive) in PBS followed by a 30 minute incubation with 100 μl fluorescein isothiocyanate-conjugated rabbit anti-mice IgG diluted 1:10. The oocytes will be washed and scored for surface zona fluorescence.

Sperm-binding assay: Based on Hewitt and England (1997) and Parish et al., (1988) protocols for oocyte in vitro maturation will be followed. Mice ovaries will be obtained from ovaries of untreated females and will be transported in PBS supplemented with 100 IU penicillin per ml and 50 mg streptomycin per ml at 39° C. Ovaries will be placed in modified TCM 199 supplemented with 0.3% BSA and will be processed within 2 h. Recovered oocytes will be washed in 3 changes of culture medium and examined by stereoscope. Oocytes completely surrounded by layers of cumulus cells will be selected and cultured for 40, 72 or 96 h at 39° C. in an humidified environment at 5% CO₂ in air. Afterwards, the excess of cumulus cells will be removed by repeated aspiration through a glass pasteur pipette. The cumulus denuded but zona covered eggs will be treated with 100 μl of the serum from immunized bitches positive to PZP antibodies at different dilutions for 1 h, washed 3 times with TL HEPES and transferred to culture dishes containing canine (or feline) sperm in TL to a final concentration of 2.5 million spermatozoa/ml; in 0.5 ml of fertilization media. The combined gametes will be incubated overnight at 39° C. in 5% CO₂ atmosphere in air. Afterwards, evaluation with a light microscopy for penetration of the oocyte will be scored as complete with one or more spermatozoa traversing completely through the zona to the perivitelline space.

Immunocytochemistry: Based on Conley, et al., (unpublished) protocol, mice ovarian tissue sections will be fixed in 4% paraformaldehyde and placed in paraffin blocks and cut into 4 μm sections and mounted on glass slides. Tissue sections will be deparafinized and hydrated through xylene and alcohol. Several washes (5 mm) with PBS and incubation will continue at the end of each of the following steps: 1) Inactivation of the endogenous peroxidases with 0.3% H₂O₂ in methanol (30 min). 2) Blocking of the nonspecific protein adhesion using 1.5% of goat serum in PBS. 3) Incubation (1 h) with 100 μl of the primary antibody (anti PZP) raised in dog. 4) Incubation (30 min) with 100 μl of the secondary antibody (biotinylated goat anti-dog). 5) Incubation (30 min) with ABC reagent (Avidin Biotin Complex). 6) Incubation (10-30 mm) with peroxidase substrate solution (AEC) until intensity of color is developed. 7) Rinsing with tap water and counterstaining with Hematoxylin. 8) Mounting with cover slip (crystal mount).

Histopathology: Histological sections will be prepared by fixing injected muscles, ovaries and uterus in 4% paraformaldehyde, embedding them in paraffin and staining sections of 4 μm with Haematoxylin and Eosin.

Results

Western Blot Analysis

Use of transfected baby hamster fibroblasts kidney cells (BHKC) with pND+PZP-3a plasmid and reacting with positive serum from dogs injected with crude PZP protein revealed a band size of 35 Kda corresponding to the deglycosilated PZP-3a protein, 55 kda corresponding to the glycosylated P2P.3α and P2P.3β protein (Yurewicz et al., 1994).

Elisa: There is no significant difference in the antibody titer levels between ID and IM administration route. Neither were there any significant differences between prolonged and sustained as reported for other nucleic acid vaccines.

Histopathology: During the experiment 3 animals died following retroorbital bleeding. Two received the vector (control) and another, the ZP-NAV (pND+PZP-3a). The mouse receiving the ZP-NAV demonstrated a reduced number of oocytes and the few (3) that were found were degenerated. The two control mice showed between 17-19 oocytes that have maintained their cellular integrity and no signs of lesions or inflammation were evident within the ovarian stroma.

Ovarian Cyclicity: Pap smears revealed that mice are continuing to cycle and some are in diestrus or anestrus.

### EXAMPLE 2

The previous example shows that DNA vaccination produced two different types of infertility. Injection of any bacterial plasmid DNA produced a transient infertility in mice, which lasts 3 to 4 months. After this time, the mice regained normal fertility. In contrast, injection of a plasmid containing a zona pellucida gene produced a long term, permanent infertility.

Materials and Methods:

Immunostimulatory sequences (ISS-ODN) and control or mutated (M1-ODN and M2-ODN) phosphorothioate were provided by Dr. E. Raz (University of California at San Diego). The compound labeled ISS-ODN actively stimulates the immune system while the other two are inactive do not affect immunity. The sequences of these oligonucleotides is shown below.

| | |
|---|---|
| ISS-ODN | ^{5′} TCA TTG GAA AAC GTT CTT CGG ^{3′} |
| (active): | |
| M1-ODN | ^{5′} TGA CTG TGA ACC TTA GAG ATG A ^{3′} |
| (inactive): | |
| M2-ODN | ^{5′} TGA CTG TGT CTC TTA GAG ATG A ^{3′} |
| (inactive): | |

All compounds were dissolved in normal saline in a final volume of 100 μl.

Experiment 1

The first experiment was designed to test whether the oligonucleotides would produce infertility and to see if there was any difference between the immunologically active and inactive oligonucleotides. Three groups of female mice tested. Group 1 was inoculated with μg of ISS-ODN (active) by single intradermal (ID) injection. Group 2 was injected with 5 μg of M1-ODN (inactive) via ID and Group 3 was untreated (Table 1).

We find that both ISS-ODN (active) and M1-ODN (inactive) affect fertility as early as the first week after inoculation. The infertility persists for many months but the animals appear to be regaining fertility by the final time point at 38.4 weeks (see Table 1). This outcome contrasts with one obtained for mice injected with pND-NP plasmid where recover fertility between 12 to 17 weeks after inoculation. An explanation for this is that the oligonucleotides used in this experiment are phosphorothioate DNA analogues and are therefore less vulnerable to nuclease degradation and most probably persist longer in the animal after injection.

Infertilities were induced by both immunologically active and inactive oligonucleotides. Uninjected mice were always fertile. These results suggest that the transient infertility induced by plasmid DNA or these oligonucleotides is not is not caused by activation of the immune system. Rather, these compounds appear to be acting directly on the reproductive system to produce their effect.

The estrous cycle appears to be normal in the infertile mice. Histological examination of the ovaries shows no difference between injected and uninjected animals or between injected animals in the infertile state compared to animals which have recovered fertility. Thus, these compounds do not appear to be toxic.

We draw the following conclusions from this experiment
1. Infertility can be induced by injection of small synthetic phosphorothioate oligonucleotides.
2. Infertility lasts longer with the phosphorothioate DNA analogues when compared to plasmid DNA

3. Both immunology active and inactive oligonucleotides produce infertility. This implies that infertility is not caused by immune activation.

TABLE 1
Effect of Intradermally Injected Phosphorothioate
Oligonucleotides on Fertility in Female Mice

| | Dose | | Pregnancy Rate | | | |
|---|---|---|---|---|---|---|
| Group | (μg) | Route | 1 week | 8 weeks | 20 weeks | 38.4 weeks |
| ISS-ODN | 5 | ID | 1/5 | 2/5 | 1/5 | 2/5 |
| M1-ODN | 5 | ID | 2/5 | 2/5 | 2/5 | 3/5 |
| Uninjected | none | none | 5/5 | 5/5 | 5/5 | 4/5 |

Experiment 2

Dose and the route of administration are variables that influence the effectiveness of vaccines and drugs. We used intraperitoneal injections rather than intradermal injection in this experiment. Dosage was also lowered from 5 to 2 μg. Groups were organized as follows: Group 1 was inoculated with a single intradermal (IP) injection of 2 μg of ISS-ODN, Group 2 was immunized with 2 μg of M1-ODN via IP and Group 3 with M2-ODN via IP. Mice were challenged for mating at different time points (Table 2).

Outcomes from this experiment were comparable to those obtained in Experiment 1. In both cases, both immunologically active and inactive oligonucleotides produced infertility. Most mice have recovered their fertility by 18 weeks after inoculation. It is possible that this difference can be explained by the different routes of injection but more likely that it is due to the different dose (2 μg versed 5 μg). In either case, it is possible to change the duration of infertility by changing the route and dose.

TABLE 2
Effect of Intraperitoneal Injection of Phosphorothioate
Oligonucleotides on Fertility in Female Mice

Pregnancy Rate

| Group | Dose (μg) | Route | 1 week | 18 weeks |
|---|---|---|---|---|
| ISS-ODN | 2 | IP | 1/4 | 3/4 |
| M1-ODN | 2 | IP | 2/4 | 4/4 |
| M2-ODN | 2 | IP | 2/4 | 3/4 |

Experiment 3

Oral administration of drugs makes them simple and convenient to use. Since phosphorothioate nucleotides are more resistant to degradation, we examined whether oral administration would be effective. In general, oral administration of drugs requires higher doses than parenteral delivery and thus we used higher amounts of oligonucleotides (a total of 120 μg/mouse) were used in this experiment. Two groups of mice were used. Group 1 received 40 μg/day for 3 days of ISS-ODN. Group 2 received M-ODN. All ISS were dissolved in normal saline in a final volume of 100 μl and fed to the animals by dropper. Mice were bred at 4 days and 18 weeks after the initial day of treatment.

Contraception was observed in both groups. The infertile state persists for at least 18 weeks in both groups (Table 3). Thus, oligonucleotides injected via ID, IP or delivered orally and have comparable contraceptive results. The fact that infertility persists for 18 weeks suggest that absorption by the digestive system may be efficient and that lower doses will also be effective.

TABLE 3
Effect of Oral Delivery of Phosphorothioate
Oligonucleotides on Fertility in Female Mice

Pregnancy Rate

| Group | Dose (μg) | Route | 4 days | 18 weeks |
|---|---|---|---|---|
| ISS-ODN | 40/day/3 days | oral | 1/5 | 1/5 |
| M1-ODN | 40/day/3 days | oral | 1/5 | 2/5 |

### Conclusion/Summary

We found that all the ISS sequences we tested produced transient infertility. These studies yield the following pertinent results:
1. All oligonucleotides tested produced transient contraception. This included sequences that are active in stimulating innate immunity as well as sequences, which do not activate an immune response. Thus, it appears that the transient contraception is not immune mediated at all. Our current hypothesis is that DNA and its analogues act directly on some component of the reproductive system to turn it off.
2. Both systemic DNA sequences and phosphothioate analogues produced transient contraception. The period of contraception for the analogues was significantly longer (7-9 months) than the normal DNA (3-4 months) and may also depend on dose.
3. The phosphothioate DNA analogues are active when given orally.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

| LOCUS | PIGZP3A 1699 bp mRNA MAM 07-OCT-1994 |
|---|---|
| DEFINITION | Sus scrofa zona pellucida sperm-binding glycoprotein |
| | (ZP3-alpha) mRNA, complete cds. |
| ACCESSION | L11000 |
| VERSION | L11000.1 GI:294237 |
| KEYWORDS | sperm-binding glycoprotein. |
| SOURCE | Sus scrofa. |
| ORGANISM | Sus scrofa |
| | Eukaryota; Metazoa; Chordata; Craniata; Vertebrata; |
| | Euteleostomi; Mammalia; Eutheria; Cetartiodactyla; Suina; |
| | Suidae; Sus. |
| REFERENCE | 1 (bases 1 to 1699) |
| AUTHORS | Yurewicz, E.C., Hibler, D., Fontenot, G.K., Sacco, A.G. and |
| | Harris, J. |
| TITLE | Nucleotide sequence of cDNA encoding ZP3 alpha, a sperm- |
| | binding glycoprotein from zona pellucida of pig oocyte |
| JOURNAL | Biochim. Biophys. Acta 1174 (2), 211-214 (1993) |
| MEDLINE | 93363643 |
| FEATURES | Location/Qualifiers |
| source | 1..1699 |
| | /organism=“Sus scrofa” |
| | /db_xref=“taxon:9823” |
| | /tissuetype=“ovary” |
| gene | 38..1648 |
| | /gene=“ZP3-alpha” |
| CDS | 38..1648 |
| | /gene=“ZP3-alpha” |
| | /function=“sperm-binding protein” |
| | /note=“precursor protein” |
| | /codon_start=1 |
| | /product=“zp3-alpha sperm-binding glycoprotein” |
| | /protein id=“AAA50164.1” |
| | /db_xref=“GI:294238” |
| /translation=“MWLRPSIWLCFPLCLALPGQLQPKAADDLGGLYCGPSSFHFSIN | |
| LLSQDTATPPALVVWDRRGRLHKLQNDSGCGTWVHKGPGSSMGVEASYRGCYVTEWDS | |
| HYLMPGILEEADAGGHRTVTETKLFKCPVDFLALDVPTIGLCDAVPVWDRLPCAPPPI | |
| TQGECKQLGCCYNSEEVPSCYYGNTVTSRCTQDGHFSIAVSRNVTSPPLLWDSVHLAF | |
| RNDSECKPVMETHTFVLFRFPFSSCGTAKRVTGNQAVYENELVAARDVRTWSHGSITR | |
| DSIFRLRVSCIYSVSSSALPVNIQVFTLPPPLPETHPGPLTLELQIAKDERYGSYYNA | |
| SDYPVVKLLREPIYVEVSIRHRTDPSLGLHLHQCWATPGMSPLLQPQWPMLVNGCPYT | |
| GDNYQTKLIPVQKASNLLFPSHYQRFSVSTFSFVDSVAKQALKGPVYLHCTASVCKPA | |
| GAPICVTTCPAARRRRSSDIHFQNGTASISSKGPMILLQATRDSSERLHKYSRPPVDS | |
| HALWVAGLLGSLIIGALLVSYLVFRKWR″ | |
| sig peptide | 38..100 |
| | /gene=“ZP3-alpha” |
| | /note=“zona pellucida sperm-binding glycoprotein signal” |
| mat peptide | 101..1645 |
| | /gene=“ZP3-alpha” |
| | /product=“zp3-alpha sperm-binding glycoprotein” |
| polyA signal | 1670..1675 |
| BASE COUNT | 385 a 478 c 417 g 419 t |
| ORIGIN | |
| 1 gaattccggg tggaagtacc tgttctccgc aggcgctatg tggttgcggc cgtccatctg | |
| 61 gctctgcttt ccgctgtgtc ttgctctgcc aggccagtct cagcccaaag cagcagatga | |
| 121 ccttggtggc ctctactgtg ggccaagcag ctttcatttc tccataaatc ttctcagcca | |
| 181 ggacacagca actcctcctg cactggtggt ttgggacagg cgcgggcggc tgcacaagct | |
| 241 gcagaatgac tctggctgtg gcacgtgggt ccacaagggc ccaggcagct ccatgggagt | |
| 301 ggaagcatcc tacagaggct gctatgtgac tgagtgggac tctcactacc tcatgcccat | |
| 361 tggacttgaa gaagcagatg caggtggaca cagaacagtc acagagacga aactgtttaa | |
| 421 gtgccctgtg gatttcctag ctcttgatgt tccaaccatt ggcctttgtg atgctgtccc | |
| 481 agtgtgggac cgattgccat gtgctcctcc acccatcact caaggagaat gcaagcagct | |
| 541 tggctgctgc tacaactcgg aagaggtccc ttcttgttac tatggaaaca cagtgacctc | |
| 601 acgctgtacc caagatggcc acttctccat cgctgtgtct cgcaatgtga cctcacctcc | |
| 661 actgctctgg gattctgtgc acctggcctt cagaaatgac agtgaatgta aacctgtgat | |
| 721 ggaaacacac acttttgtcc tcttccggtt tccatttagt tcctgtggga ctgcaaaacg | |
| 781 ggtaactggg aaccaggcgg tatatgaaaa tgagctggta gcagctcggg atgtgaggac | |
| 841 ttggagccat ggttctatta cccgagacag catcttcagg cttcgagtca gttgtatcta | |
| 901 ctctgtaagt agcagtgctc tcccagttaa catccaggtt ttcactctcc caccaccgct | |
| 961 tccggagacc caccctggac ctcttactct ggagcttcag attgccaaag atgaacgcta | |
| 1021 tggctcctac tacaatgcta gtgactaccc ggtggtgaaa ttgcttcggg agcccatcta | |
| 1081 tgtggaggtc tctatccgtc accgaacaga ccccagtctc gggctgcacc tgcaccagtg | |
| 1141 ctgggccaca cccggcatga gccccctgct ccagccacag tggcccatgc tagtcaatgg | |
| 1201 atgcccctac actggagaca actaccagac caaactgatc cctgtccaga aagcctcaaa | |
| 1261 cctgctattt ccttctcact accagcgttt cagtgtttcc accttcagtt ttgtggactc | |
| 1321 tgtggcaaag caggcactca agggaccggt gtatctgcat tgtactgcat cggtctgcaa | |
| 1381 gcctgcaggg gcaccgatct gtgtgacaac ctgtcctgct gccagacgaa gaagaagttc | |
| 1441 tgacatccat tttcagaatg gcactgctag catttctagc aagggtccca tgattctact | |
| 1501 ccaagccact cgggactctt cagaaaggct ccataaatac tcaaggcctc ctgtagactc | |
| 1561 ccatgctctg tgggtggctg gcctcttggg aagcttaatt attggagcct tgttagtgtc | |
| 1621 ctacctggtc ttcaggaaat ggagatgagt tactcagacc aaatgtgtca ataaaaccaa | |
| 1681 taaaacaaaa ccggaattc | |
| // | |
| LOCUS | PIGZP3B 1326 bp mRNA MAM 04-AUG-1993 |
| DEFINTION | Pig zona pellucida glycoprotein mRNA, complete cds. |
| ACCESSION | L22169 |
| VERSION | L22169.1 GI:347422 |
| KEYWORDS | zona pellucida glycoprotein. |
| SOURCE | Sus scrofa ovary cDNA to mRNA. |
| ORGANISM | Sus scrofa |
| | Eukaryota; Metazoa; Chordata; Craniata; Vertebrata; |
| | Euteleostomi; Mammalia; Eutheria; Cetartiodactyla; Suina; |
| | Suidae; Sus. |
| REFERENCE | 1 (bases 1 to 1326) |
| AUTHORS | Yurewicz, E.C., Hibler, D., Fontenot, G.K. and Harris, J. |
| TITLE | Cloning and sequence analysis of cDNA encoding ZP3-beta of pig |
| | oocyte zona pellucida |
| JOURNAL | Unpublished |
| FEATURES | Location/Qualifiers |
| source | 1..1326 |
| | /organism=“Sus scrofa” |
| | lab_xref=“taxon:9823” |
| | /tis sue type=“ovary” |
| sig peptide | 25..81 |
| | /note=“putative” |
| CDS | 25..1290 |
| | /standard_name=“ZP3-beta” |
| | /note=“homologue of mouse ZP3; putative” |
| | /codon_start=1 |
| | /product=“zona pellucida glycoprotein” |
| | /protein_id=“AAA31145.1” |
| | /db_xref=“GI:347423” |
| /translation=″MAPSWRFFVCFLLWGGTELCSPQPVWQDEGQRLRPSKPPTVMVE | |
| CQEAQLVVIVSKDLFGTGKLIRPADLSLGPAKCEPLVSQDTDAVVRFEVGLHECGSSL | |
| QVTDDALVYSTFLRHDPRPAGNLSILRTNRAEVPIECHYPRQGNVSSWAILPTWVPFR | |
| TTVFSEEKLVFSLRLMEENWSAEKMTPTFQLGDRAHLQAQVHTGSHVPLRLFVDHCVA | |
| TLTPDWNTSPSHTIVDFHGCLVDGLTEASSAFKAPRPGPETLQFTVDVFHFANDSRNT | |
| IYITCHLKVTPADRVPDQLNKACSFSKSSNRWSPVEGPAVICRCCHKGQCGTPSLSRK | |
| LSMPKRQSAPRSRRHVTDEADVTVGPLIFLGKTSDHGVEGSTSSPTSVMVGLGLATVV | |
| TLTLATIVLGVPRRRRAAAHLVCPVSASQ″ | |
| polyA signal | 1286..1291 |
| | /note=“putative” |
| BASE COUNT | 265 a 403 c 388 g 270 t |
| ORIGIN | |

```
      1 gaattccggg gccttgtgag tgccatggcg ccgagctgga ggttcttcgt ctgctttctg
    61 ctctggggag gtacagagct atgcagcccg cagcccgtct ggcaggacga aggccagcgc
  121 ttgaggccct caaagccacc caccgtaatg gtggagtgtc aggaggccca gctggtggtc
  181 attgtcagca aagacctttt cggtaccggg aagctcatca ggcctgcaga tctcagcctg
  241 ggccctgcaa agtgtgagcc gctggtctct caggacacgg acgcagtggt caggtttgag
  301 gttgggctgc acgagtgtgg cagcagcttg caggtgactg atgatgctct ggtgtacagc
  361 accttcctgc gccatgaccc ccgccctgca ggaaacctgt ccatcctgag gacgaaccgt
  421 gcggaggtcc ccatcgagtg tcactacccc aggcagggca acgtgagcag ctgggccatc
  481 ctgcccacct gggtgccctt caggaccacg gtgttctccg aggagaagct ggtgttctct
  541 ctgcgcctga tggaggaaaa ctggagtgcc gagaagatga cgcccacctt ccagctgggg
  601 gacagagccc acctccaggc ccaagtccac accggcagcc acgtgccact gaggctgttt
  661 gtggaccact gtgtggccac gctgacgccg gactggaaca cctccccctc tcacaccatc
  721 gtggacttcc acggctgtct cgtggacggt ctcactgagg cctcatctgc tttcaaagca
  781 cctagacctg gaccagagac gctccagttc accgtggatg tgttccattt tgctaatgat
  841 tccagaaaca cgatctacat cacctgccat ctgaaggtca ctccggctga ccgagtcccg
  901 gaccaactca acaaagcctg ttccttcagc aagtcctcca acaggtggtc cccggtggaa
  961 gggcctgctg ttatctgtcg ttgctgtcac aaggggcagt gtggtacccc aagcctttcc
1021 aggaagctgt ctatgccgaa gagacagtct gctccccgca gtcgcaggca cgtgacagat
1081 gaagcagatg tcacagtggg gcctctgatc ttcctgggca agacgagtga ccacggtgtg
1141 gaagggtcca cctcctcccc cacctcggtg atggtgggct tgggcctggc caccgtggtg
1201 accttgactc tggctaccat tgtcctgggt gtgcccagga ggcgtcgggc tgctgcccac
1261 cttgtgtgcc ccgtgtctgc ttcccaataa aaggagaaac atgaaaaaaa aaaaaaaccg
1321 gaattc
//
```

1. A method for inhibiting fertility in a female mammal in need thereof comprising administering to the mammal a pharmaceutical composition comprising an isolated nucleic acid in an amount sufficient to inhibit fertility in the mammal.

2. The method of claim 1, wherein the nucleic acid is an oligonucleotide.

3. The method of claim 1, wherein the nucleic acid comprises an immunostimulatory sequence.

4. The method of claim 3, wherein the pharmaceutical composition does not comprise an antigen against which a protective immune response is desired.

5. The method of claim 1, wherein the mammal is a human.

6. The method of claim 1, wherein the mammal is a dog.

7. The method of claim 1, wherein the pharmaceutical composition is administered intradermally.

8. The method of claim 1, wherein the pharmaceutical composition is administered orally.

9. The method of claim 1, wherein the nucleic acid is a phosphorothioate analogue.

10. The method of claim 1, wherein the amount sufficient to induce infertility is a dose between about 50 μg/kg and about 1 mg/kg.
